(19) United States
(12) Patent Application Publication
(10) Pub. No.: US 2026/0183562 A1
(43) Pub. Date: Jul. 2, 2026

(54) PHOTODYNAMIC THERAPY OF TUMOR BY ILLUMINATION FROM AN ENDOLUMINAL CAVITY

(71) Applicants: YEDA RESEARCH AND DEVELOPMENT CO. LTD., Rehovot (IL); MEMORIAL SLOAN KETTERING CANCER CENTER, New York, NY (US); IMPACT BIOTECH LTD, Ness Ziona (IL)

(72) Inventors: Avigdor SCHERZ, Tel Aviv (IL); Yaniv COHEN, Ma'ale Adumim (IL); Dina PREISE, Ashdod (IL); Hooman YARMOHAMADI, New York, NY (US); Stephan SOLOMON, New York, NY (US); Zachary SACKS, Modiin (IL); David KELSEN, New York, NY (US); Franz Edward BOAS, New York, NY (US); Keren SASSON, Rehovot (IL); Lilach AGEMY, Herzliya (IL)

(21) Appl. No.: 19/132,248

(22) PCT Filed: Nov. 27, 2022
(86) PCT No.: PCT/US2022/051023

§ 371 Date: May 22, 2025

Related U.S. Application Data
(60) Provisional application No. 63/427,867, filed on Nov. 24, 2022.

(51) Int. Cl. A61N 5/06; A61M 25/00; A61M 25/01; A61M 25/10; A61N 5/00; A61N 5/067

(52) U.S. Cl. CPC A61N 5/062; A61M 25/10; A61N 5/0601; A61N 5/067; A61M 2025/0004; A61M 25/0108; A61N 2005/005; A61N 2005/063; A61N 2005/0664

(57) ABSTRACT

Intraluminal photodynamic therapy (PDT) of a target tumor tissue that is carried out from within a blood vessel is disclosed. The PDT method may be a vascular-targeted photodynamic therapy (VTP) or any other form of PDT where photosensitizer concentration in the blood vessel endothelium is not phototoxic. The PDT of this disclosure is carried out from within a blood vessel in the vicinity of the target tissue or organ with a light-diffusing section of an irradiating optical fiber being positioned within the confines of an inflatable balloon section of a balloon catheter, which is inflated during irradiation to occlude blood flow.

### TECHNOLOGICAL FIELD

The presently disclosed subject matter is in the general field of photodynamic therapy (PDT) and more particularly in the field of vascular-targeted photodynamic therapy (VTP).

### BACKGROUND ART

Photodynamic therapy (PDT) is a form of phototherapy involving a dynamic interaction between a photosensitizer drug, oxygen, and light to elicit cell death. In classical PDT, photosensitizer drugs administered to a subject may preferentially accumulate in the tumor tissues, because of the enhanced permeabilization and retention (EPR) effect and thereafter undergo further accumulation in the rapidly proliferating cancer cells. Photosensitizer activation by application of a light dose at specific wavelengths limited to the tumor site, generates short-living reactive oxygen species (ROS) by interaction with local oxygen that results in apoptosis or necrosis of the cancer cells. Most currently used sensitizers are activated at the time of preferential accumulation in the tumor tissue and uptake by the cancer cells. Furthermore, the active ROS generated by these sensitizers is singlet oxygen, which oxidizes proteins and lipids essential to cell survival (Type II mechanism), thereby leading to cell death by apoptosis or necrosis and consequently tumor non-thermal ablation.

Vascular-targeted photodynamic therapy (VTP) is a photodynamic therapeutic technique whereby photosensitization is applied shortly after intravenous (IV) administration of the photosensitizer, while still in the circulation to a large extent. Some VTP drugs such as Redaporfin or Visudyne (Verteporfin) are delivered in liposomes and rapidly taken up by the vascular endothelial cells. Subsequent tumor illumination causes endothelial cell death, vascular thrombosis, and flow arrest. In contrast, bacteriochlorophyll derivatives (Bchl-D), such as Padeliporfin (also known by its code name WST11), are water soluble VTP drugs that non-covalently bind to serum albumin and are neither taken up by the tumor endothelium, nor extravasate until clearance. Illumination of the tumor vascular bed by light at the range of red to near infrared-light, delivered through an optical fiber, typically connected to a laser, activates the circulating Bchl-D. The activated Bchl-D generates hydroxyl and superoxide radicals (Type I mechanism) followed by local hypoxia and endogenous nitric oxide generation. A cascade of biological processes is initiated resulting in tumor vascular arrest followed by destruction and cell killing that culminates in a coagulative necrosis of the tumor tissue, while normal blood vessels, particularly those larger than 40 microns in diameter, can be preserved. The endothelial and cancer cell necrosis is followed by activation of anti-tumor immunity that completes the eradication of the illuminated tumor. WST11-VTP has been recently termed Padeliporfin ImPACT (Immune Photo Activated Cancer Therapy).

### GENERAL DESCRIPTION

This description concerns intraluminal photodynamic therapy (PDT) of a target tumor tissue that is carried out from within a blood vessel, typically a major blood vessel, in the vicinity of the tumor. By one preferred embodiment of this disclosure, the PDT method is a vascular-targeted photodynamic therapy (VTP). Other exemplary embodiments are immune photo-activated cancer therapy (ImPACT) and a PDT that targets a photosensitizer drug absorbed by the cancer cells.

The term “PDT” as used herein is meant to denote all these types of PDT, preferably VTP. Also, in the description below, reference is made, at times, to PDT, which is one specific embodiment of this disclosure, it being understood that it is not meant to be limiting of the disclosure, which applies to the full scope as noted above.

The terms “PDT drug”, “PDT-effective drug”, “photosensitizer drug”, or the like, are meant to denote one or a combination of agents that are administered to the subject and that can be activated by light to cause them to generate a chemically reactive species, typically, but not exclusively, a reactive oxygen species.

The terms “PDT-effective light”, “photosensitizing-effective light”, or the like, are meant to denote light that has the effect of activating the PDT-effective drug.

The term “about” denotes a quantity which may deviate by up to 10%, 15%, or even 20%, from the stated quantity.

It was realized, in accordance with this disclosure, that while light delivery through an optical fiber positioned within the endoluminal cavity of large blood vessels, including arteries or other tubular organs, can be safely used for initiating PDT, particularly, but not exclusively, in VTP of tumors involving major and accessible vessels the absorption of light by blood born elements, such as hemoglobin (that peaks at 780 nm), by the circulating photosensitizer drug may considerably attenuate the emitted light, which may be a limiting factor for such therapeutic treatment. To be more specific, the attenuation of light that reaches the walls of the blood vessel would be roughly proportional to the vessel's diameter and, accordingly, there would be an inverse relationship between this diameter and the amount of light that would reach the tumor tissue that surrounds such vessels. Some of the light may be absorbed by the circulating photosensitizer drug: the thicker the blood layer between the fiber and the vessel's wall—the higher the attenuation (Beer-Lambert rule or light diffuser model, if including scattering), and lower light fluency is left to exit the vessel's wall as needed for activation of the photosensitizer drug in the tumor, particularly the VTP-effective photosensitizer in the tumor's vasculature. It was further realized, in accordance with this disclosure, that such attenuation could be avoided by temporarily occluding blood from the irradiated section of the blood vessel, by means of a balloon catheter, and irradiating the drug sensitizing-effective light from within the inflatable balloon section thereof while it is inflated. In this manner, the irradiated light would reach the walls of the blood vessel with only small attenuation, passing through the inflatable balloon section that is made of transparent or translucent material, and from there passing on through the blood vessel's walls to the tumor. In this manner, also, any negative effect that may otherwise occur from an interaction between the light and the photosensitizer drug that circulates also through the vessel from which light irradiation is made, would be avoided; with such an interaction occurring only outside this vessel.

In other words, by the present disclosure the blood layer between the light-diffusing element at the distal end of the irradiating optical fiber and the wall of the vessel, that would otherwise optically separate said element from the vessel walls and, hence, reduce irradiation damage and possibly induce some local harm within the vessel, is safely thinned or removed in the procedure provided in this disclosure, whereby the light reaching the tumor through the vessel's wall will cause ablations of tumors surrounding such vessels.

Provided by this disclosure is a catheter system and a catheter assembly assembled out of elements of the catheter system useful in a PDT procedure, a therapeutic system that comprises the catheter system or assembly, and a PDT method. All these are independent aspects of this disclosure. As can be appreciated, embodiments that are described with reference to one aspect may be applicable also in one or more of the others, as can no doubt be appreciated by the skilled reader.

Provided by one aspect of this disclosure is a catheter system, that comprises an optical fiber, a balloon catheter, and a flushing channel each of which axially extends between respective proximal and distal ends.

The optical fiber is optically couplable at its proximal end to a light source, typically a light source that emits a PDT-effective light (at PDT-effective wavelengths, which depend on the nature of the photosensitizer drug that is being used, and at sufficient intensity). It further has a light-diffusing section (that may also be referred to, interchangeably, as “light diffuser”) that is, typically, cylindrical, at a distal end portion configured for scattering light transmitted through the fiber such that at least a portion thereof is transmitted from said light diffuser in a non-axial direction; particularly in a radial direction. Fitted on the fiber, at said end portion, are one or more fiber-associated imaging markers that, as will be further explained below, are useful (under appropriate imaging) to track the location of the light-diffusing section and, thus, ensure that it is properly positioned before light irradiation is initiated. The distal marker further provides an indication of the location of the fiber tip within the balloon catheter, especially with regards to extending beyond the distal end of the flushing channel.

A variety of light diffusers may be used in accordance with this disclosure. These include diffusers that scatter light substantially equally in all radial directions, front-face diffusers emitting a circular beam, and side-firing diffusers in which the beam is directed to only one side. In addition, the term “diffuser” in this disclosure may also refer to other light discharging elements at the fiber's distal end that discharge the light, including clear-cut fibers (polished or cleaved) that discharge light as a Gaussian or super-Gaussian beam, conical dischargers in which the discharged light pattern is a ring centered around the fiber in a forward direction, and a lensed fiber emitting a beam with larger divergence in the forward direction. In the case of other light emitters, scattering may refer to ray light propagation determined through surfaces, reflective, or refractive media.

The balloon catheter is fluidically couplable at its proximal end to a source of pressurized fluid (that may be a liquid or a gas and which may contain an imaging contrast material) and has an inflatable balloon section at its distal end portion. Fitted on the catheter are one or more balloon-associated imaging markers that are associated with the inflatable balloon section and that, similarly to the fiber-associated imaging markers, are useful to track the location of the inflatable balloon section and, thus, ensure that it is properly positioned before light irradiation is initiated both with regards to the location in the vessel and the location of the optical fiber's light-diffusing section within the balloon. The balloon catheter, as known per se, also has a working channel.

The flushing channel may be constituted by said working channel (a typical, although not exclusive, embodiment) or by a separate tube that may be fitted within, and which extends through the balloon catheter's working channel. The flushing channel is fluidically couplable to a source of flushing physiological solution (the term “flushing physiological solution”, interchangeable with “flushing solution”, is meant to encompass any compatible physiological fluid, including a saline solution, a saline solution supplemented with other components, etc.) at its proximal end. This is intended for cooling the optical fiber's light-diffusing section that would otherwise heat up during light irradiation, prevent blood from flowing into the open working channel where the fiber is located, which is required to remain open in order to allow balloon placement using a guidewire, and to a lesser extent, separate the highly absorbing blood from the diffuser at the very distal end of the system or assembly disclosed herein. The flushing channel extends between a liquid-sealed proximal end and an open distal end, whereby the flushing solution is permissible to flow along the flushing channel and to exit it at the distal end into the artery distally to the inflatable balloon section of the balloon catheter. The flushing channel is dimensioned to permit it to accommodate the optical fiber that is introduced through the liquid-sealed proximal end, and to permit a flushing solution to flow along the fiber's entire introduced length between the flushing channel's proximal and distal ends.

The imaging markers, by one embodiment, are X-ray markers, which may be configured as annular metallic elements, or in certain cases as a metal rod, for example near the distal tip of the fiber. However, as can be appreciated by other embodiments, the imaging markers may be differently configured, including polymer-based X-ray markers, or adapted to other imaging techniques, such as ultrasound-based imaging.

The inflatable balloon section has, typically, walls that are transparent or translucent to the irradiating light and may be adapted to permit passage therethrough of at least 50% of the light that impinges thereon.

The system, by some embodiments, comprises an auxiliary catheter that is fluidically couplable at its proximal end to an auxiliary liquid source, typically a contrast saline solution (namely saline with a contrast agent, whereby the contrast agent may constitute about 10% of the total solution) that may be used as an imaging aid for positioning the inflatable balloon section at the intended distal location, and potentially as a means to provide a fluid barrier between the balloon and the blood in the artery at the balloon's proximal end. It has a catheter lumen that extends between a liquid-sealed proximal end and an open distal end. The lumen is dimensioned to allow it to accommodate the balloon catheter that is introduced through the liquid-sealed proximal end and to permit the auxiliary liquid to flow from the proximal to the distal end even when accommodating the balloon catheter. The auxiliary catheter has a length such that when accommodating the balloon catheter, the distal end of the balloon catheter, including the inflatable balloon section, extends out of the distal portion of the auxiliary catheter.

It should be noted that a contrast agent may, by some embodiments of this disclosure, also be incorporated into the flushing solution.

There may be, typically (albeit not exclusively), one or two fiber-associated imaging markers and one or two balloon-associated imaging markers. The imaging markers may be positioned at a defined position—the at least one fiber-associated imaging marker at a defined position with respect to the light-diffusing section and the at least one balloon-associated imaging marker at a defined position with respect to the inflatable balloon section. The defined position is typically one close to the proximal or distal end of the respective section. In some embodiments there may be two fiber-associated imaging markers and/or two balloon-associated imaging markers. In the case of a set of two such markers, they would typically flank the respective section. The respective markers are useful for imaging-based position guidance to ensure that the light diffuser of the optical fiber is axially positioned within the inflatable balloon section of the balloon catheter. Where there are two fiber-associated imaging markers flanking the light-diffusing section and two balloon-associated imaging markers flanking the inflatable balloon section, the axial distance between the two former markers and that between the two latter markers may be different to permit a comprehensive view of them all, without the two of one set overlapping the other. In other words, the distance D1 between the two fiber-associated imaging markers and the distance D2 between the two balloon-associated imaging markers should be different, D1 being typically shorter than D2. However, it is possible also for D1 and D2 to be substantially the same, whereby proper positioning of the light-diffusing section vis-à-vis the inflatable balloon section is ensured by ‘merging’ the images of the two sets. The light-diffusing section is usually shorter than the inflatable balloon section to permit its positioning within the axial limits of the inflatable balloon section.

A catheter assembly is another aspect of this disclosure and comprises elements of the catheter system assembled together and co-axially extending in a proximal to distal direction. All embodiments described above are also applicable with respect to the assembly.

A therapeutic system is another aspect of this disclosure. It comprises the catheter system described that may be assembled together to form said assembly or may comprise the already pre-assembled catheter assembly.

The therapeutic system also comprises a light source for emitting light at a suitable wavelength, particularly visible, NIR, or SWIR that is configured for coupling to the optical fiber's proximal end. This range may encompass light in the range of about 350 nm to 1600 nm, particularly within the range of about 400 nm to 800 nm. The light source is typically (albeit not exclusively) adapted to emit light of a narrow bandwidth, e.g., a laser. Where the photosensitizer drug is a bacteriochlorophyll derivative, a suitable laser is one emitting light at a central wavelength of about 750-756 nm, with a bandwidth typically less than 4 nm. The power output of the laser light entering the optical fiber through its proximal end should, typically, be up to about 2 Watts.

The therapeutic system may also comprise a source of contrast saline solution fluidically couplable to the auxiliary catheter's proximal end. Furthermore, a contrast agent may also be comprised in the flushing solution and in the balloon-inflation fluid. The therapeutic system disclosed herein may also comprise a photosensitizer drug.

A method for photodynamic therapy (PDT) of a target site within a subject is a further aspect of this disclosure. It comprises the following elements in the stated order or in any other suitable order. For example, the systemic administration of the photosensitizer drug described under (a) may be prior, during or after the insertion of the assembly described under (b), and may also even be during the treatment inducing stage described under (c).

The method includes the following elements:
(a) Systemically administering a photosensitizer drug to a subject. This may be achieved through intravenous administration, but also by other means. The photosensitizer drug may, for example, be a bacteriochlorophyll derivative that can absorb and be sensitized by light at a wavelength of about 750-756 nm. A particular example of such a photosensitizer drug is Padeliporfin (known also as WST11). Another example is Verteporfin that is sensitized by light at a wavelength of about 680-700 nm or Redaporfin that is sensitized by light at a wavelength of about 740-760 nm. Other non-limiting examples include Photofrin, Phtalocyanine, ALA-Ppix, Talaprofin, or Temoporfin.
(b) Inserting an assembly as described herein through a blood vessel and guiding it until the inflatable balloon section of the balloon catheter is in the vicinity of the target site, typically tangential to the target tissue, e.g., a tumor, and axially displacing the optical fiber to position the light-diffusing section within said inflatable balloon section. Optionally, this element may also comprise adjusting the balloon size and location using standard radiological techniques, inflating the balloon, and introducing imaging contrast material through the auxiliary catheter thereafter, etc. It should be noted that rather than inserting the assembly in an assembled form, it may also be formed in situ, for example, by first inserting said auxiliary catheter, then inserting the balloon catheter from its proximal end through the lumen of the auxiliary catheter, and then inserting the optical fiber through the balloon catheter's working channel. A typical insertion uses a guidewire placed in the balloon's working channel. Once the balloon is placed in the proper location, the guidewire is removed from the working channel and the fiber is inserted.
(c) Inducing a treatment stage that comprises inflating the inflatable balloon section to substantially occlude blood flow through the blood vessel, e.g., by inflating the inflatable balloon section until the walls of the inflatable balloon section come into contact with those of the blood vessel, which also leads to centering the fiber within the vessel. This stage further comprises inserting the fiber with the light-diffusing section to position using the imaging markers, passing a flushing solution through the flushing channel, and irradiating light through the optical fiber at the wavelength and intensity suitable for activating the photosensitizer drug. The treatment stage may be performed for a time period between about 1, 2, 3 or 4 min. and about 6, 7, 8, 9, 10, 11, or 12 min; about 5 min. being a specific example. By some embodiments, treatments up to 20 or even 30 minutes are also possible.

In an embodiment using Padeliporfin, balloon placement (b) typically occurs before drug infusion (a).

The method is typically performed while imaging at least the target site in the subject to ensure positioning of the inflatable balloon section adjacent to the target site and for axially positioning the light-diffusing section within said inflatable balloon section through proper alignment of the one or more fiber-associated imaging markers with the one or more balloon-associated imaging markers.

Where the fiber comprises a distal marker, the fiber distal marker may be placed directly underneath the distal marker of the balloon or at close proximity thereto.

Where the fiber comprises a set of two fiber-associated imaging markers flanking the light-diffusing section and axially separated from one another by a distance D1, and the balloon catheter comprises a set of two balloon-associated imaging markers flanking the inflatable balloon and axially separated from one another by a distance D2, different than D1, for said proper alignment the optical fiber is axially displaced until one of the sets flanks the other. Where D2 is larger than D1, then for said proper alignment the optical fiber is axially displaced until the two fiber-associated imaging markers are flanked by the two balloon-associated imaging markers.

The flushing saline may be flushed continuously through the flushing channel throughout the PDT procedure to ensure that the optical fiber, and particularly its light-diffusing section, does not heat excessively.

The PDT method of this disclosure is typically a VTP method for initiating tumor ablation by fast occlusion and destruction of the tumor-associated vasculature. An example is pancreatic cancer, wherein the distal end of the catheter assembly is guided into the superior mesenteric artery (SMA), superior mesenteric vein (SMV), celiac artery or into any other large vessel accessible for catheterization in the vicinity of the pancreas.

An exemplary treatment regime is one comprising two or more repeated treatment stages (as described under (a) above) separated by rest stages, in which the inflatable balloon section is deflated thereby eliminating light transmission outside the vessel and/or light irradiation is stopped for a certain time. The rest stage is typically between about 0.5 or 1 min. and about 1.25, 1.5, 1.75 or 2 min. ; about 1 min. being a specific example.

Other exemplary indications using the system or assembly described herein include angiosarcoma; abdominal paraaortic lymph nodes with cancer involvement; treatments within and surrounding the heart or other locations accessible by a blood vessel, including the brain, kidney, liver, lung, neck, muscle, and connective tissue; or treatments in the gallbladder or other hollow cavity with a fluid to be displaced. In addition to ablation, other treatments enabled by this delivery method may include changes in tissue properties such as stiffening, or a localized chemical reaction due to light activation.

### EMBODIMENTS

Some non-limiting embodiments of this disclosure are described in the following numbered paragraphs. These embodiments are intended to add on to, and not limit, the disclosure of the above general description.
1. A catheter system, comprising an optical fiber, a balloon catheter, and a flushing channel each of which axially extends between respective proximal and distal ends;
   the optical fiber
      being optically couplable at its proximal end to a light source,
      having a light-diffusing section at a distal end portion configured for scattering light transmitted through the fiber such that at least a portion thereof is transmitted from said light diffuser in a non-axial direction, and
      having one or more fiber-associated imaging markers at said end portion;
   the balloon catheter
      being fluidically couplable at its proximal end to source of pressurized fluid,
      having an inflatable balloon section at its distal end portion,
      having one or more balloon-associated imaging markers associated with the inflatable balloon section, and
      having a working channel; and
   the flushing channel
      being fluidically couplable to a source of flushing solution at its proximal end,
      extending between a liquid-sealed proximal end and an open distal end,
      being capable of accommodating the optical fiber introduced through the liquid-sealed proximal end and dimensioned to permit a flushing solution to flow along the fiber's entire introduced length between the flushing channel's proximal and distal ends, and
      being constituted by said working channel or by a separate tube that may be fitted and extends through the balloon catheter's working channel.
2. The system of embodiment 1, wherein the flushing channel is said balloon working channel.
3. The system of embodiment 1 or 2, wherein the imaging markers are X-ray markers.
4. The system of embodiment 3, wherein the imaging markers are annular metallic elements or circular elements.
5. The system of any one of embodiments 1 to 4, wherein the inflatable balloon section has transparent or translucent walls.
6. The system of embodiment 5, wherein said walls permit passage therethrough of at least 50% of the light that impinges thereon.
7. The system of any one of embodiments 1 to 6, comprising an auxiliary catheter
   being fluidically couplable at its proximal end to a liquid source,
   having a catheter lumen extending between a liquid-sealed proximal end and an open distal end, capable of accommodating the balloon catheter introduced through the liquid-sealed proximal end, and being dimensioned to permit the liquid to flow from the proximal to the distal end, even when accommodating the balloon catheter, and
   having a length such that when accommodating the balloon catheter, the distal end of the balloon catheter, including the inflatable balloon section, extends out of the distal section of the auxiliary catheter.
8. The system of any one of embodiments 1 to 7, comprising at least one fiber-associated imaging marker at a defined position with respect to the light-diffusing section.
9. The system of embodiment 8, comprising a fiber-associated imaging marker near the proximal or distal end of the light-diffusing section, typically, at a known distance.
10. The system of embodiment 8, comprising two fiber-associated imaging markers flanking the light-diffusing section.
11. The system of any one of embodiments 1 to 10, comprising at least one balloon-associated imaging marker at a defined position with respect to the inflatable balloon section.
12. The system of embodiment 11, comprising a balloon-associated imaging marker near the proximal or distal end of the inflatable balloon section.
13. The system of any one of embodiments 1 to 10, comprising two balloon-associated imaging markers flanking the inflatable balloon section.
14. The system of any one of embodiments 8 to 13, comprising
   a fiber-associated imaging marker at a defined position with respect to the light-diffusing section; and
   two balloon-associated imaging markers flanking the inflatable balloon section.
15. The system of embodiment 14, comprising one fiber-associated imaging marker at a proximal end of the light-diffusing section.
16. The system of any one of embodiments 1 to 15, comprising
   two fiber-associated imaging markers flanking the light-diffusing section and axially separated from one another by a distance D1, and
   two balloon-associated imaging markers flanking the inflatable balloon section and axially separated from one another by a distance D2, different than D1.
17. The system of embodiment 16, wherein D2 is larger than D1.
18. The system of any one of embodiments 1 to 17, wherein
   the light-diffusing section is shorter than the inflatable balloon section, and wherein
   the optical fiber is axially displaceable within the flushing channel to axially align the light-diffusing section to be within axial confines of the inflatable balloon section.
19. The system of any one of embodiments 1 to 18, for use in PDT, e.g., VTP (including immune photo-activated cancer therapy, i.e., ImPACT), or PDT/VTP that targets the tumor parenchyma.
20. The system of any one of embodiments 1 to 19, wherein the optical fiber has a coupling member at its proximal end configured for optical coupling of said proximal end to a light source emitting light at a therapeutically effective wavelength.
21. A catheter assembly, comprising an optical fiber, a balloon catheter with a working channel, and a flushing channel all axially extending in a proximal to distal direction and having each, respective, proximal and distal ends.
   the balloon catheter
      being fluidically couplable at its proximal end to a source of pressurized fluid,
      having an inflatable balloon section at its distal end portion,
      having one or more balloon-associated imaging markers associated with the inflatable balloon section, and
      having a working channel;
   the flushing channel
      being fluidically couplable to a source of flushing solution at its proximal end,
      extending between a liquid-sealed proximal end and an open distal end,
      accommodating the optical fiber introduced through the liquid-sealed proximal end and being dimensioned to permit a flushing solution to flow around the fiber's entire introduced length between the flushing channel's proximal and distal ends, and
      being constituted by said working channel or by a tube accommodated within and extending through said working channel or being constituted by a lumen of a tube accommodated within the working channel; and
   the optical fiber
      being optically couplable at its proximal end to a light source,
      having a light-diffusing section at a distal end portion configured for scattering light transmitted through the fiber such that at least a portion thereof is transmitted from said light diffuser in a non-axial direction,
      having one or more fiber-associated imaging markers at said end portion of the flushing channel, and
      being accommodated within the flushing channel in a manner permitting the flushing solution to flow from the flushing channel's proximal to its distal end along the fiber's entire introduced length.
22. The assembly of embodiment 21, wherein the flushing channel is said working channel.
23. The assembly of embodiment 21 or 22, wherein the imaging markers are X-ray markers.
24. The assembly of embodiment 23, wherein the imaging markers are annular or circular metallic elements.
25. The assembly of any one of embodiments 21 to 24, wherein the inflatable balloon section has transparent or translucent walls.
26. The assembly of embodiment 25, wherein said walls permit passage therethrough of at least 50% of the light that impinges thereon.
27. The assembly of any one of embodiments 21 to 26, comprising an auxiliary catheter
   being fluidically couplable at its proximal end to a liquid source
   having a catheter lumen extending between a liquid-sealed proximal end and an open distal end, capable of accommodating the balloon catheter introduced through the liquid-sealed proximal end and being dimensioned to permit the liquid to flow from the proximal to the distal end, even when accommodating the balloon catheter, and
   having a length such that the distal end of the balloon catheter, including the inflatable balloon section, extends out of the distal section of the auxiliary catheter.
28. The assembly of any one of embodiments 21 to 27, comprising at least one fiber-associated imaging marker at a defined position with respect to the light-diffusing section.
29. The assembly of embodiment 28, comprising a fiber-associated imaging marker near the proximal or distal end of the light-diffusing section.
30. The assembly of embodiment 29, comprising two fiber-associated imaging markers flanking the light-diffusing section.
31. The assembly of any one of embodiments 21 to 30, comprising a balloon-associated imaging marker at a defined position with respect to the inflatable balloon section.
32. The assembly of embodiment 31, comprising a balloon-associated imaging marker near the proximal or distal end of the inflatable balloon section.
33. The assembly of any one of embodiments 21 to 32, comprising two balloon-associated imaging markers flanking the inflatable balloon section.
34. The assembly of any one of embodiments 21 to 33, comprising
   a fiber-associated imaging marker at a defined position with respect to the light-diffusing section; and
   two balloon-associated imaging markers flanking the inflatable balloon section.
35. The assembly of embodiment 34, comprising one fiber-associated imaging marker at a proximal end of the light-diffusing section.
36. The assembly of any one of embodiments 21 to 35, comprising
   two fiber-associated imaging markers flanking the light-diffusing section and axially separated from one another by a distance D1, and
   two balloon-associated imaging markers flanking the inflatable balloon section and axially separated from one another by a distance D2, different than D1.
37. The system of embodiment 36, wherein D2 is larger than D1.
38. The assembly of any one of embodiments 21 to 37, wherein
   the light-diffusing section is shorter than the inflatable balloon section, and wherein
   the optical fiber is axially displaceable within the flushing channel to axially align the light-diffusing section to be within axial confines of the inflatable balloon section.
39. The assembly of any one of embodiments 21 to 38, for use in PDT e.g., VTP (including immune photo-activated cancer therapy, i.e., ImPACT), or PDT/VTP that targets the tumor parenchyma.
40. The assembly of any one of embodiments 21 to 39, wherein the optical fiber has a coupling member at its proximal end configured for optical coupling of said proximal end to a light source emitting light at a therapeutically effective wavelength (e.g., effective to activate a photosensitizer drug).
41. A therapeutic system comprising the catheter system of any one of embodiments 1 to 20 or the assembly of any one of embodiments 21 to 40.
42. The system of embodiment 41, comprising a light source for emitting light at an ultraviolet, visible, NIR, and SWIR range (e.g., light within the range of about 350 nm to 6500 nm), configured for coupling to the optical fiber's proximal end.
43. The system of embodiment 41 or 42, wherein the light source emits light of a narrow bandwidth.
44. The system of embodiment 43, wherein the light source is a laser.
45. The system of any one of embodiments 41 to 44, comprising
   an auxiliary catheter according to embodiment 7 or 27, and
   a source of contrast saline solution fluidically couplable to the auxiliary catheter's proximal end.
46. The system of any one of embodiments 41 to 45, comprising a photosensitizer drug.
47. A method for photodynamic therapy (PDT) of a target site within a subject, comprising
   systemically administering (e.g., intravenously) to the subject a photosensitizer drug;
   inserting an assembly of any one of embodiments 19 to 36 through a blood vessel and axially advancing and guiding it until the inflatable balloon section of the balloon catheter is in the vicinity of and, typically, tangential to the target site and axially displacing the optical fiber to position the light-diffusing section within said inflatable balloon section;
   inducing a treatment stage, comprising inflating the inflatable balloon section, passing a flushing solution through the flushing channel, and irradiating light through the optical fiber at a wavelength and intensity suitable for activating the photosensitizer drug.
48. The method of embodiment 47, comprising
   imaging the target site, and
   axially positioning the light-diffusing section within said inflatable balloon section through proper alignment of the one or more balloon-associated imaging markers with the one or more fiber-associated imaging markers.
49. The method of embodiment 47, wherein
   The optical fiber comprises a set of two fiber-associated imaging markers flanking the light-diffusing section and axially separated from one another by a distance D1, and
   the balloon catheter comprising a set of two balloon-associated imaging markers flanking the inflatable balloon section and axially separated from one another by a distance D2, different than D1, and wherein
   for said proper alignment the optical fiber is axially displaced until one of the sets flanks the other.
50. The method of embodiment 49, wherein
   D2 is larger than D1, and wherein
   for said proper alignment the optical fiber is axially displaced until the two fiber-associated imaging markers are flanked by the two balloon-associated imaging markers.
51. The method of any one of embodiments 47 to 50, comprising
   continuously flushing a flushing physiological solution through the flushing channel throughout the PDT procedure.
52. The method of any one of embodiments 47 to 51, wherein
   the PDT is vascular-targeted photodynamic therapy (VTP).
53. The method of embodiment 50, wherein
   the light source is a laser emitting light at a wavelength of about 750-756 nm with a power output of up to about 2 Watts.
54. The method of embodiment 52 or 53, wherein
   the photosensitizer drug is a bacteriochlorophyll derivative having a major light absorption at about 750-756 nm and capable of generating oxygen radicals upon illumination, and wherein
   said photosensitizer drug is intravenously administered to the subject for a defined time period before inflation of the inflatable balloon section and initiating light irradiation.
55. The method of embodiment 52 or 53, wherein said photosensitizer drug is Padeliporfin, Verteporfin or Redaporfin.
56. The method of any one of embodiments 47 to 55, wherein the photosensitizer drug is administered for about 5 -15 min. (typically about 10 min.) prior to light irradiation.
57. The method of any one of embodiments 47 to 55, wherein the photosensitizer drug is administered for about 5-30 min. (typically about 10 min.) prior to light irradiation.
58. The method of any one of embodiments 47 to 57, wherein
   the target site is a tumor, and wherein
   the blood vessel is a major vessel passing in the vicinity of the tumor.
59. The method of embodiment 58, wherein
   the tumor is pancreatic cancer, and wherein
   the blood vessel is the superior mesenteric artery (SMA), or superior mesenteric vein (SMV), or any other large vessel accessible for catheterization in the vicinity of the pancreas.
60. The method of embodiment 58 or 59, wherein the blood vessel is substantially tangential to the tumor.
61. The method of any one of embodiments 47 to 60, comprising
   two or more treatment stages separated by rest stages, as needed, in which the inflatable balloon section is deflated (light irradiation may or may not be discontinued during the rest stage).
62. The method of embodiment 61, wherein
   the treatment stages are for a period of about 2 to about 10 min., and wherein
   the rest stages are for about 0.5 to about 2 min.
63. The method of any one of embodiments 47 to 62, wherein
   the inflatable balloon section is inflated to substantially occlude blood flow through the blood vessel.
64. The method of any one of embodiments 47 to 63, wherein the inflatable balloon section is inflated to substantially occlude blood flow through the blood vessel and center the fiber within said vessel.
65. The method of embodiments 63 or 64, wherein the inflatable balloon section is inflated such that its walls come into contact with those of the blood vessel.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a general schematic representation of a catheter assembly according to an exemplary embodiment of this disclosure deployed in the body for the performance of VTP in the pancreas.

FIG. 1B depicts the distal portion of the assembly with respect to its target site.

FIG. 1C is a detailed schematic representation of a catheter system that includes the assembly, and portions thereof, depicted in FIGS. 1A and 1B.

FIG. 2 presents a schematic, albeit more realistic, depiction of the ex vivo elements of the assembly of the system of FIG. 1C.

FIG. 3 is a schematic representation of the distal portion of the assembly of FIGS. 1A-2.

FIG. 4 shows a flow chart of the deployment of the assembly and of the performance of a VTP procedure, according to an exemplary embodiment of this disclosure.

FIG. 5 shows a schematical representation of the distal portion of the assembly, according to an embodiment of a VTP treatment regime of this disclosure, in several operational states.

FIG. 6 is a graphical legend to explain the shadings that are used for the different elements in FIG. 5.

FIG. 7A and FIG. 8B show images captured during real-time fluoroscopy for, respectively, guiding the inflatable balloon section of the catheter and of the light diffuser into proper alignment within the confines of the balloon catheter, making use of the imaged balloon-associated imaging markers and the fiber-associated markers.

FIG. 8 is a schematic illustration showing the sites along the external femoral artery wall (proximal and distal) where light intensity was measured, using isotropic probes, to assess effective light irradiation during the VTP procedure.

FIG. 9 is a histological image of pancreatic and other tissues following a VTP procedure as disclosed herein.

### DETAILED DESCRIPTION OF SPECIFIC EMBODIMENTS

In the following description exemplary embodiments are described that focus primarily on the VTP aspect of this disclosure. As can be appreciated, the described embodiments are non-limiting examples only of the full scope of this disclosure defined and described above and in the appended claims.

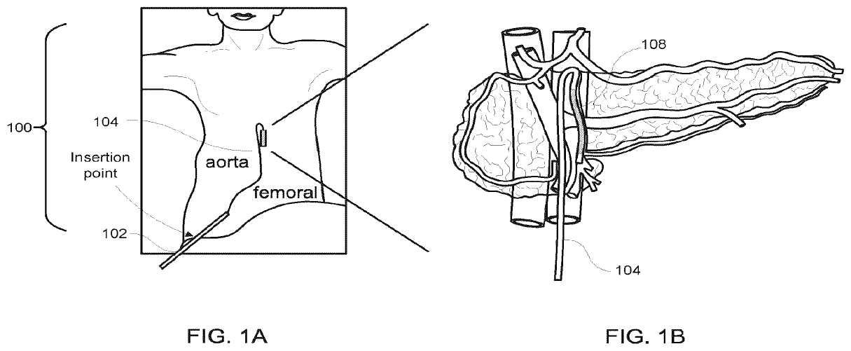

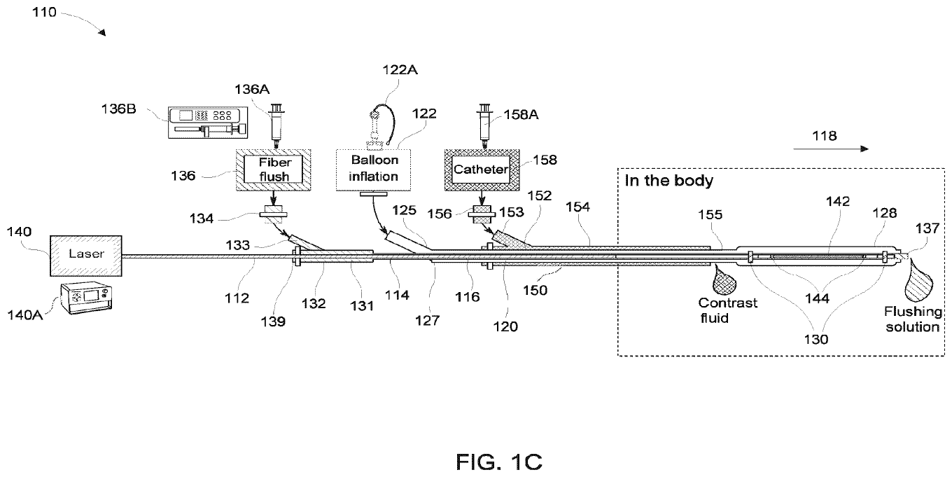

Referring first to FIGS. 1A and 1B, shown is a catheter assembly, generally designated 100 that includes a proximal portion 102 outside the body, and a distal portion, of which only the optical fiber 104 is illustrated, within the body. The assembly is inserted through the body wall at the insertion point into the femoral artery and then extended into the abdominal aorta with its terminal, light-diffusing section 108 being bent (bending diameter may be less than 25 mm) and introduced into the superior mesenteric artery that lies over the pancreas.

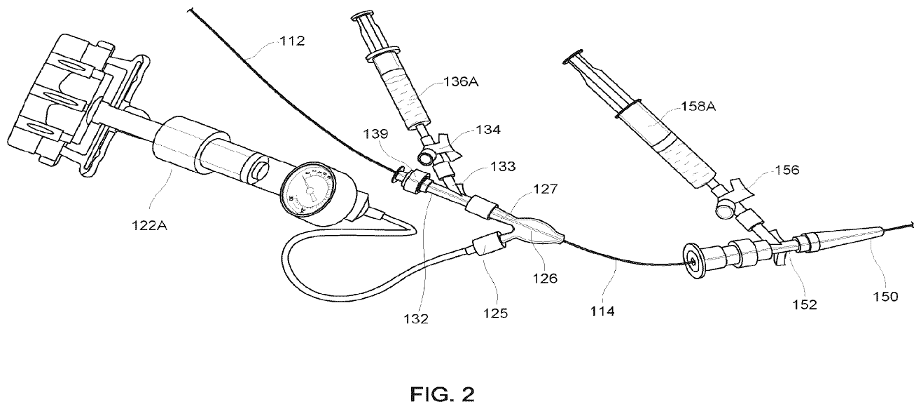

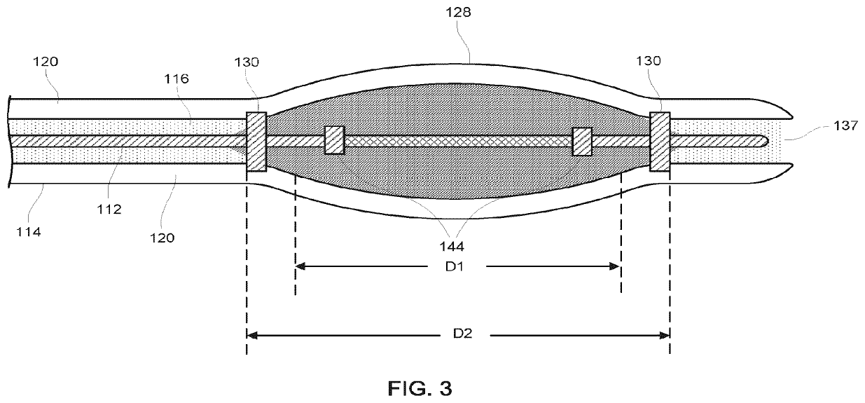

Reference is now being made to FIG. 1C, FIG. 2 and FIG. 3, wherein: FIG. 1C is a schematic illustration of a catheter system, generally designated 110 that includes the catheter assembly 100 and other system elements, all being schematically illustrated; FIG. 2 being a more realistic depiction of elements of the assembly of FIG. 1C that remain outside the body; and FIG. 3 being a schematic representation of the distal end portion of the assembly.

The catheter system 110 of this embodiment includes an optical fiber 112 and a balloon catheter 114 with a working channel 116, all axially extending in a proximal to distal direction, along an axis (which in use assumes a tortious path) represented by arrow 118 and between, respective, proximal and distal ends.

The balloon catheter 114 (which may be, for example, a Powerflex™ or a Saber™, Cordis Medical, USA) has a pressurized fluid conduit 120 that envelopes the working channel 116 and is fluidically coupled to a source of pressurized fluid 122 (which may, or may not, comprise a contrast agent), such as a manually operated device 122A (for example, IN4130-BasxCOMPAK™, Merit Medical, USA) through a port 125 of a balloon Y-connector 126. Another port 127 of the balloon Y-connector is coupled to the outlet port 131 of the hemostasis Y-connector 132 to be described below. The balloon catheter 114 has an inflatable balloon section 128 at its distal end portion.

Flanking the working region of the inflatable balloon section 128 and situated within the balloon volume are annular elements formed on the inner wall of the balloon, comprising a pair of balloon-associated imaging markers 130 that are, in this specific example, X-ray markers and therefore visible through X-ray imaging for gauging the position of the balloon. The two markers 130 are separated from one another by a certain distance D2. It should be noted that in other embodiments of this disclosure there may be only one such marker, associated with either the proximal or distal end of the balloon section, or there may also be more than two such markers. The X-ray markers 130 may be made of metal, e.g., gold, or a metal alloy.

Formed within the balloon catheter, as noted above, is a working channel 116 that serves also as a flushing channel, the function of which will be described below. The terms “working channel” and “flushing channel” may be used interchangeably in the description below regarding this embodiment. It should be noted that in other embodiments of this disclosure the flushing channel may be a dedicated conduit accommodated within the working channel.

The flushing channel 116 is fluidically coupled at its proximal end with an outlet port 131 of a hemostasis Y-connector 132, a port 133 thereof being coupled, through a coupling element 134 (which may be a controllable or a one-way valve) to a source 136 of a flushing physiological solution (which may, by some embodiments, comprise a contrast agent), which may be a manually operated syringe 136A or an automated pump 136B. The working channel 116 has an opening 137 at its distal end and hence the fluid within this channel is maintained at a pressure similar to that of the average blood pressure (and in fact slightly higher to permit the flushing solution to flow within the flushing channel from the liquid-sealed proximal to the open distal end and to prevent backflow of blood). The hemostasis Y-connector 132 has a liquid-sealed proximal end (achieved using a Tuohy-Borst adapter that prevents fluid leakage) configured to permit introduction, through the proximal end, of the optical fiber 112 (for example, CDS-23S, LifePhotonics GmbH, Germany) in a fluid-tight manner to maintain pressure of the flushing solution. The optical fiber 112 is dimensioned to permit the flushing solution to flow around the fiber's entire introduced length between the flushing channel's proximal and distal ends. The typical (but not exclusive) outer diameter of the fiber is 0.75 mm, while the diameter of the balloon working channel is 0.89 mm, thereby allowing the placement of the fiber within the balloon and flushing of the flushing solution throughout. In one use, the light-diffusing section 142 (see below) of the optical fiber 112 can heat up considerably and the continuous flushing of the flushing solution that egresses out of the distal open end 137 of the working channel 116 serves for significantly cooling the light-diffusing section of the optical fiber, preventing blood backflow, and hydrating the vessel.

The optical fiber 112 is optically couplable at its proximal end to a light source 140, typically a laser device 140A (for example, a ML7710, Modulight Inc., Finland). The optical fiber 112 has a light-diffusing section 142 at a distal end portion that is configured for scattering light transmitted through the fiber such that at least a portion thereof is transmitted from said light diffuser in a non-axial direction, particularly radially. Associated with and flanking the light-diffusing section are two imaging markers, typically X-ray markers 144. These X-ray markers 144 are separated from one another by a certain distance D1, different and shorter than D2 in this embodiment. This facilitates proper relative positioning of said light diffuser vis-à-vis the inflatable balloon section.

The inflatable balloon section 128 is typically made from a transparent material but may be, by some embodiments, translucent to thereby permit passage therethrough of most of the light that is irradiated from the light-diffusing section 142.

The system 110 also has an auxiliary catheter 150, which may be a cobra catheter, with a hemostatic Y-connector 152 at its proximal end. Here, also, a Tuohy-Borst adapter may be used to prevent fluid leakage. It has a catheter lumen 154 that extends between a liquid-sealed proximal end, that is defined by the Y-connector 152 and an open distal end 155 and accommodates the balloon catheter, introduced through the liquid-sealed proximal end. The catheter lumen 154 is dimensioned to permit the fluid to flow from the proximal to the distal end. The lumen 154 is coupled, through port 153 of the Y-connector 152 and a coupling element 156 to a source of contrast fluid 158, which may be a manually operated syringe 158A filled with a contrast agent, typically, but not exclusively, dissolved in saline. This contrast fluid that egresses out of the open distal end 155 of catheter 150 aids in the positioning of the auxiliary catheter and the associated proper positioning of the balloon catheter. The auxiliary catheter 150 has, as can be appreciated, a length that is less than that of the balloon catheter 114 that extends out of the distal end 155 of the auxiliary catheter 150.

It should be noted that in certain uses and applications of this disclosure a contrast agent may also be included in the balloon inflating liquid and/or in the flushing solution.

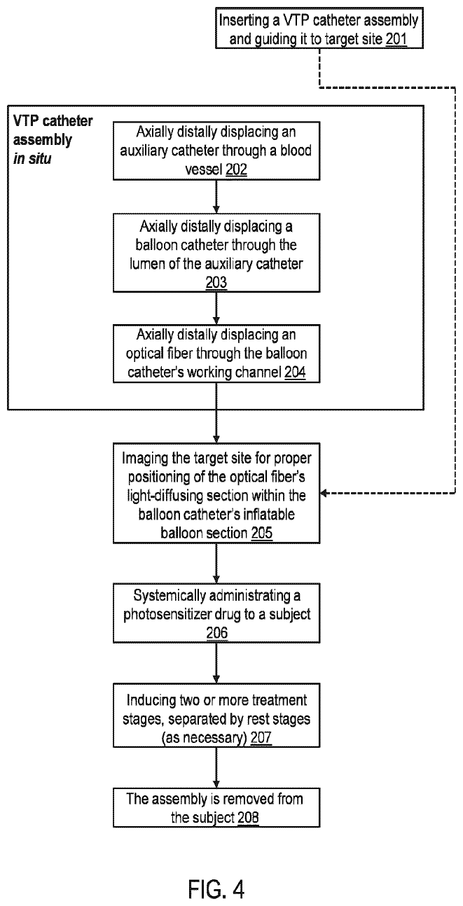

Reference is now being made to FIG. 4, which is a non-limiting example of a VTP procedure of a target site within a subject. It comprises procedure steps 201-208 which may be carried out in the described order or, by some other embodiments, in different, suitable orders. For example, the systemic administration of the photosensitizer drug of step 206 may be prior, during or after the deployment of the assembly in step 201 or assembling its elements in situ, as described in steps 202-204, and may also be during step 205 when aligning the light-diffusing section within the inflatable balloon section, and may even be during treatment step 207. Thus, as can be understood, the VTP procedure embodied in FIG. 4 is but a non-limiting example.

By one embodiment, the assembly is assembled ex vivo and then deployed in an assembled form. This is represented by step 201 that comprises inserting a VTP assembly, as disclosed herein, through a blood vessel and guiding it until the inflatable balloon section of the balloon catheter is in the vicinity of the target site. Then, as represented by step 205, under imaging, the optical fiber's position may be adjusted to ensure proper placement of the fiber's light-diffusing section within the confines of the inflatable balloon section of the balloon catheter.

Alternatively, rather than deploying the assembly in its assembled form, it may also be assembled in situ, for example in the manner exemplified in steps 202-204. By this example, an auxiliary catheter is first inserted into the respective blood vessel and is positioned, with the optional aid of a contrast agent, as exemplified by step 202. In the next step 203 the balloon catheter is inserted through the proximal end of the catheter's lumen and is axially and distally displaced and positioned such that its distal inflatable balloon section is at the intended treatment site. In the subsequent step 204, the optical fiber is inserted through the balloon catheter's working channel and axially distally displaced. The next step can be step 205 as already discussed above.

A guidewire may be used for the positioning of the auxiliary catheter and/or the balloon catheter, as known per se. In the case of the latter, once the balloon section is properly positioned, the guidewire can be removed, and the optical fiber is then inserted.

Imaging at least the target site in the subject is performed to ensure the proper positioning of the inflatable balloon section adjacent to the target site and the proper axial positioning of the light-diffusing section of the optical fiber within the inflatable balloon section, exemplified by step 205. This imaging-guided position is aided by the imaging markers associated with the inflatable balloon section and with the light-diffusing section of the optical fiber, detailed in FIG. 3 above. In the case of the non-limiting and exemplary embodiment in which the balloon catheter has two balloon-associated markers flanking the inflatable balloon section and the optical fiber has two fiber-associated markers flanking the light-diffusing section, the axial distance D1 between the two latter markers should be different and is typically shorter than the distance D2 between the two balloon-associated markers, thus permitting a comprehensive view of all markers without any one set overlapping the other. Proper alignment of the light-diffuser section within the inflatable balloon section would be achieved once the fiber-associated markers are within the axial confines of the balloon-associated markers.

For proper alignment, the fiber is axially displaced, under imaging, until the fiber-associated markers are positioned axially within the confines of the balloon-associated markers.

In this embodiment, following proper alignment of the optical fiber's light-diffusing section within the inflatable balloon section a photosensitizer drug is systemically administered to the subject, as exemplified in step 206. This may be achieved through intravenous administration, but also by other means. The photosensitizer drug may be, for example, a bacteriochlorophyll derivative that can absorb and be sensitized by light at a wavelength of about 750-756 nm. A particular example of such a photosensitizer drug is Padeliporfin, i.e., WST11. Another example is Verteporfin that is sensitized by light at a wavelength of about 680-700 nm or Redaporfin that is sensitized by light at a wavelength of about 740-760 nm.

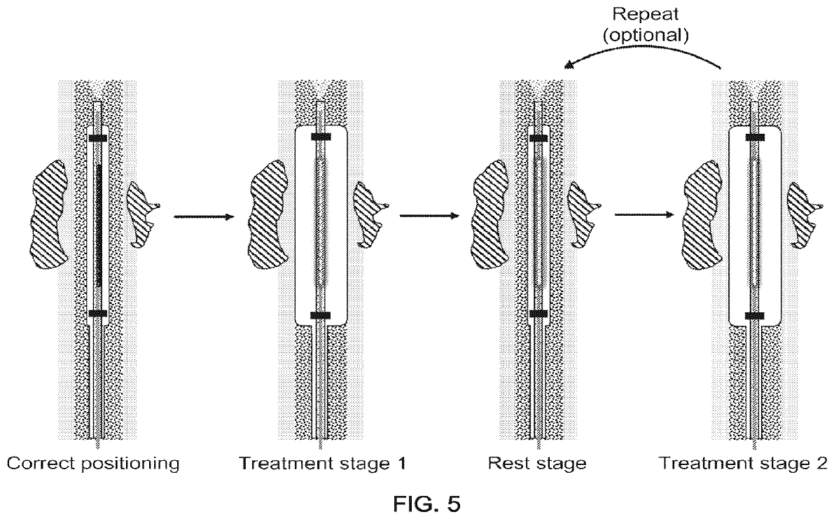

One or more treatment stages involving balloon inflation and light irradiation may then follow as suggested by step 207. Typically, there may be two or more treatment stages separated by rest stages, as needed, during which light irradiation may or may not be discontinued, and the balloon is deflated to permit blood flow. An exemplary treatment regime as disclosed in FIG. 5 may include two or more treatment stages wherein the balloon section is inflated, separated by shorter deflation rest stages, as needed. By this example, light is irradiated during the treatment stages and continues during the rest stage. In other embodiments of this disclosure, light irradiation may be discontinued during the generally shorter rest stages. Once the clinically desired treatment regime is concluded, light irradiation is ceased and the assembly herein disclosed is removed from the subject in step 208.

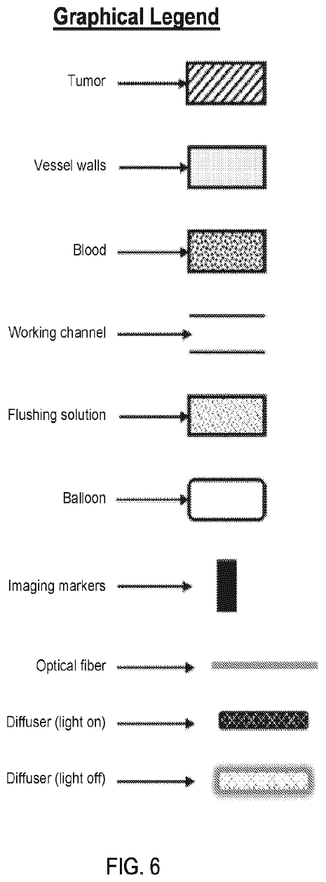

Reference is now being made to FIG. 5 and to its graphical legend in FIG. 6, which provides a schematic illustration of a non-limiting embodiment of a VTP treatment regime of this disclosure. As previously referred to in FIG. 4, a VTP procedure of this disclosure may include two or more treatment stages of balloon inflation and light irradiation. After proper positioning (far left illustration in FIG. 5), the inflatable balloon section is inflated to substantially occlude blood flow through the blood vessel (artery or vein), typically until the balloon walls come into contact with those of the blood vessel (second drawing from the left). Light is then irradiated at a wavelength and intensity suitable for activating the photosensitizer drug, while continuously flushing a flushing solution through the working channel, to thereby prevent excessive heating of the light-diffusing section and blood backflow, the flushing solution egressing through the working channel's open distal end. After a time period of several minutes, typically about 5 to 10 min., light irradiation may or may not be discontinued, while flow of the flushing solution may be ceased as well, and the balloon is deflated (third drawing from the left), permitting blood to flow through the vessel again thereby avoiding ischemic damages that may occur following prolonged blood occlusion. After a rest period, the inflatable balloon section is inflated once more and light irradiation is resumed, if it was previously discontinued, while a flushing solution continuously flows through the working channel, to continue the ablation of the vasculature at the target tissue surrounding the blood vessel. The rest period is typically between about 0.5 and 2 min.; about 1 min. being a specific example. This treatment regime of balloon inflation and light irradiation treatment stages, followed by rest stages wherein the balloon is deflated and light irradiation may or may not be discontinued, may be repeated several times, as necessary.

It should be noted that while in the disclosure herein some emphasis was made on treatment of cancer (by PDT-mediated ablation of associated vasculature) and also deployment of the disclosed assembly through an artery, this is not limiting as the assembly may also be deployed through a vein and also used within the framework of treatments other than for an indication of cancer. Additionally, the assembly of this disclosure may also be deployed via other ducts and the target organ for treatment may be other than a neoplastic tumor, for example, heart, gallbladder, urinary bladder, etc.

Additionally, in the catheters of the system of this disclosure use may be made of flow control valves or stoppers, such as one-way valves or flow control pumps.

### EXAMPLES

To demonstrate the concept of safe and feasible ablation of tissue adjacent to major blood vessels by VTP (e.g., Padeliporfin ImPACT), the following treatment protocol was devised and tested. For evaluation of preclinical safety, as described in Example 1, a variation of the proposed protocol was used, which is also detailed below.

### Materials and Methods

### Laser System Assembly

An optical fiber (LifePhotonic GmbH, Germany) was connected to a laser source (ML7710 Modulight, Finland) through a SMA connector. The laser was configured to deliver a total power of 600 mW/cm, through its diode. The output of the diode was measured with an external integrated sphere, as detailed below. The laser was set to “Ready” before light activation.

### Balloon Deployment Under Fluoroscopy

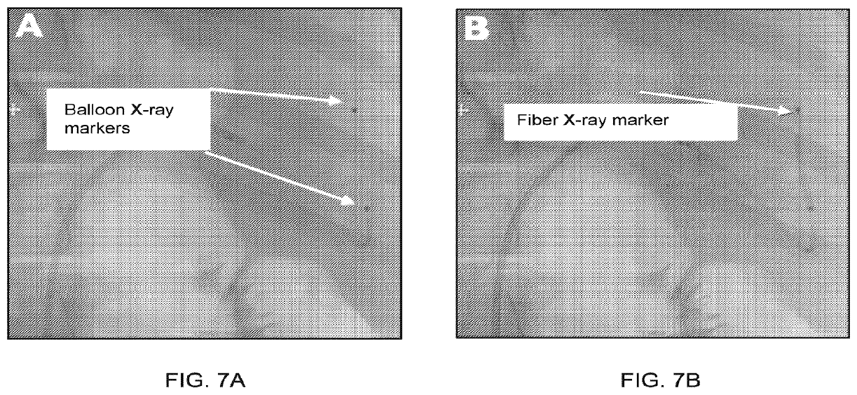

The experiment was done under the approval of Institutional Animal Care and Use Committee (IACUC) and complied with the Animal Welfare Law—Experiments in Animals 1994. A Large white/Landrace female pig (51-54 kg) was laid down at dorsal recumbency. A 6 Fr sheath was placed by the Seldinger technique in the femoral artery. Heparin was administered to the animal at a dose of 100-150 IU/kg. A 5 Fr guiding catheter (Cobra C2, Merit Medical, USA) was placed at the orifice of the celiac artery and an angiography of the arteries (celiac, splenic) was performed as a baseline and to enable measurement of the targeted artery size. An exchange guiding wire (Radiofocus™ 0.035″/260 cm, Terumo, Japan) was placed in the celiac (proximal splenic) or distal splenic arteries. The guiding catheter was withdrawn, and a Powerflex® Dilatation Catheter (Cordis Medical, USA) was placed at the proximal celiac artery or the splenic artery (being the targeted artery) and the guiding wire was withdrawn. The optical fiber was inserted through the balloon catheter lumen and positioned using the imaging markers of the balloon and fiber, as illustrated in FIGS. 7A and 7B, respectively, such that the entire light diffuser (3 cm length, 0.8 mm diameter) was nestled within the balloon (4 cm length). At the time of light activation, the balloon was inflated to reach the diameter of the artery thus obstructing blood flow and centering the fiber in the artery. Upon completion of the treatment regime, the fiber was withdrawn, and in some experimental runs the guiding catheter was replaced at the celiac orifice while a second angiography was performed to visualize and evaluate blood flow and changes, if any, to the targeted artery. The catheter and the sheath were withdrawn, and the femoral artery was closed by direct pressure for at least 20 min.

### Preparation and Administration of Padeliporfin (WST11) for Endoluminal VTP

Padeliporfin (WST11) was dissolved, under dim light, in a 5% sterile glucose solution to a concentration of 10 mg/ml prior to administration according to the manufacturer's instructions. After the optical fiber was placed in the target position, the Padeliporfin solution was infused to the animal intravenously for 10 min., using a syringe pump, to a total dose of 4 mg/kg body weight. Immediately following completion of infusion, the laser was activated for 5 min., paused for 1 min., and then reactivated for additional 5 min. (a total illumination time of 10 min.). The balloon was inflated together with laser activation and deflated when illumination was paused/completed. FIG. 5, as described above, provides a non-limiting example of a balloon inflation/deflation scheme. During illumination, sterile saline solution was continuously injected into the working channel of the balloon at a rate of ˜1.6 ml/min. Upon completion of the treatment regime, the balloon catheter and the fiber were pulled out. The procedure was performed under dim light in an operating room, from the beginning of infusion and through to completion of treatment. The animals were kept under dim light until recovery from anesthesia (˜1 hr) to ensure drug clearance. Sham control animals were subjected to balloon and fiber deployment, without infusion of Padeliporfin and consequent illumination, i.e., the balloon was inflated and deflated per the above treatment protocol and then removed.

### Necropsy and Histopathology

Necropsy was performed immediately after sacrificing the animals (KCl injection 24/96 hrs post-treatment). Gross clinical observations were recorded. Photographs were taken when deemed necessary, e.g., if gross findings were identified.

For histopathological analysis, the illuminated segment of the artery was exposed and excised together with adjacent tissue. Additional organs were collected as needed. The collected tissue was fixed in 4% formalin for at least 3-5 days. For each treated artery segment 3-4 sections were cut with ˜1 cm spacing and subjected to processing. Paraffin blocks were prepared and sections of 4-5 μm thickness were cut and stained with hematoxylin and eosin. Tissue processing was performed by a certified laboratory.

### Example 1: Preclinical Safety

First, to assess the safety of the above-described treatment protocol, intra-arterial activation of Padeliporfin was performed in 2 healthy female Yorkshire pigs (˜45 kg), using a 2 cm balloon laser catheter and 1 cm diffuser. From femoral artery access, an 8 Fr (2.36 mm) clear silicone balloon catheter was advanced into the right external iliac artery. The laser fiber was advanced into the balloon catheter. Heparin (100 IU/kg) and Padeliporfin (4 mg/kg) were infused intravenously for 10 min. The balloon was inflated with 1 ml of 50% contrast, and the laser fiber was then illuminated (753 nm, 300 mW/cm) for 20 min. The balloon was periodically deflated (1 minute for every 5 min. balloon inflation), and the catheter was flushed with saline solution to prevent clot formation and distal ischemia. Safety was evaluated by catheter angiography and necropsy post-treatment. Catheter angiography after intra-arterial VTP showed no thrombus or arterial injury. Gross pathology showed no injury to the arterial wall.

### Example 2: Preclinical Light Transmission Measurement

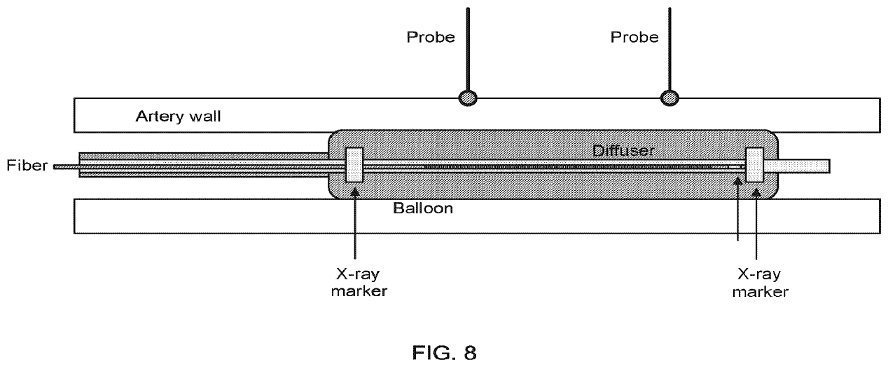

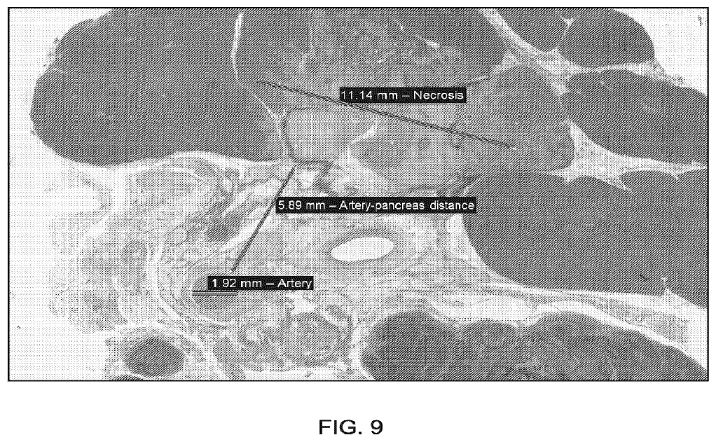

Next, the protocol described above under “Materials and methods” was performed to measure and assess effective light transmission. Thus, immediately following Padeliporfin infusion to the animal, the laser was activated at 753 nm. The light intensity on the exposed artery wall, including connective tissue, was measured using a 0.85 mm diameter spherical isotropic probe that was manually held against the external artery wall—first with the balloon deflated and then with the balloon inflated—at proximal and distal sites of the balloon, using two identical probes as illustrated in FIG. 8. Blood samples were collected for each light fluency measurement for resolving photosensitizer drug concentration during measurement. Similar measurements were taken before drug infusion as well. Results indicated up to a 10-fold increase in light intensity transmission between inflated and deflated balloon states with measured intensities reaching values of up to 740 mW/cm².

### Example 3: Preclinical Ablation Example

The treatment protocol described above under “Materials and methods” was then performed to assess its efficacy for initiating ablation of a pancreatic tumor. As described, a cylindrical diffuser (3 cm length, 0.8 mm diameter) connected to an optical fiber enabling delivery of a max power of 600 mW/cm, was incorporated into an endovascular Cordis PowerFlex Pro® PTA Dilation Catheter positioned at a selected site starting at the celiac artery and continuing to the proximal splenic artery. The site was selected based on its proximity to the pancreatic tissue. Following a 10 min. infusion of Padeliporfin the treatment protocol was initiated as described in FIG. 5, with light irradiation discontinued during the rest stage(s). The histological image selected for FIG. 9 clearly shows no damage to the vessel wall, but ablation of the pancreas, more than 5 mm away from the vessel, was observed. Other tissue structures surrounding the pancreas (e.g., nerves, lymph nodes, etc.) did not present unrecoverable changes either, thus demonstrating the selectivity and safety of the tested treatment protocol. In sham control animals, exposed to the balloon inflation/deflation scheme without administration of Padeliporfin and laser activation, pancreatic parenchyma in close proximity to the artery appeared normal. The artery wall showed mostly preserved arterial wall. Two foci of endothelial denudation with adjacent intimal necrosis were observed, possibly representing a balloon injury.

1-28. (canceled)

29. A catheter system, comprising an optical fiber, a balloon catheter, and a flushing channel each of which axially extends between respective proximal and distal ends;
the optical fiber
being optically couplable at its proximal end to a light source,
having a light-diffusing section at a distal end portion configured for scattering light transmitted through the fiber such that at least a portion thereof is transmitted from said light diffuser in a non-axial direction, and
having one or more fiber-associated imaging markers at said distal end portion;
the balloon catheter
being fluidically couplable at its proximal end to a source of pressurized fluid,
having an inflatable balloon section at its distal end portion,
having one or more balloon-associated imaging markers associated with the inflatable balloon section, and
having a working channel; and
the flushing channel
being fluidically couplable to a source of flushing solution at its proximal end,
extending between a liquid-sealed proximal end and an open distal end, and
being capable of accommodating the optical fiber introduced through the liquid-sealed proximal end and dimensioned to permit a flushing solution to flow along the fiber's entire introduced length between the flushing channel's proximal and distal ends, and
being constituted by said working channel or by a separate tube that may be fitted and extends through the balloon catheter's working channel.

30. The system of claim 29, wherein the imaging markers are X-ray markers or annular metallic elements.

31. The system of claim 29, comprising an auxiliary catheter
being fluidically couplable at its proximal end to a liquid source,
having a catheter lumen extending between a liquid-sealed proximal end and an open distal end, capable of accommodating the balloon catheter introduced through the liquid-sealed proximal end and being dimensioned to permit the liquid to flow from the proximal to the distal end even when accommodating the balloon catheter, and
having a length such that when accommodating the balloon catheter, the distal end of the balloon catheter, including the inflatable balloon section, extends out of the distal end portion of the auxiliary catheter.

32. The system of claim 29, wherein
the light-diffusing section is shorter than the inflatable balloon section, and wherein
the optical fiber is axially displaceable within the flushing channel to axially align the light-diffusing section to be within axial confines of the inflatable balloon section.

33. A catheter assembly, comprising an optical fiber, a balloon catheter with a working channel, and a flushing channel all axially extending in a proximal to distal direction and having each, respective, proximal and distal ends;
the balloon catheter
being fluidically couplable at its proximal end to a source of pressurized fluid,
having an inflatable balloon section at its distal end portion,
having one or more balloon-associated imaging markers associated with the inflatable balloon section, and
having a working channel;
the flushing channel
being fluidically couplable to a source of flushing solution at its proximal end,
extending between a liquid-sealed proximal end and an open distal end,
accommodating the optical fiber introduced through the liquid-sealed proximal end and being dimensioned to permit a flushing solution to flow around the fiber's entire introduced length between the flushing channel's proximal and distal ends, and
being constituted by said working channel or by a tube accommodated within and extending through said working channel or being constituted by a lumen of a tube accommodated within the working channel; and
the optical fiber
being optically couplable at its proximal end to a light source,
having a light-diffusing section at a distal end portion configured for scattering light transmitted through the fiber such that at least a portion thereof is transmitted from said light diffuser in a non-axial direction,
having one or more fiber-associated imaging markers at said distal end portion of the flushing channel, and
being accommodated within the flushing channel in a manner permitting the flushing solution to flow from the flushing channel's proximal to its distal end along the fiber's entire introduced length.

34. The assembly of claim 33, wherein the imaging markers are X-ray markers or annular metallic elements flanking the light-diffusing section.

35. The assembly of claim 33, comprising an auxiliary catheter
being fluidically couplable at its proximal end to a liquid source,
having a catheter lumen extending between a liquid-sealed proximal end and an open distal end, accommodating the balloon catheter with a proximal portion thereof extending out through the liquid-sealed proximal end and being dimensioned to permit the liquid to flow from the proximal to the distal end, and
having a length such that the distal end of the balloon catheter, including the inflatable balloon section, extends out of the distal end portion of the auxiliary catheter.

36. The assembly of claim 33, wherein
the light-diffusing section is shorter than the inflatable balloon section, and wherein
the optical fiber is axially displaceable within the flushing channel to axially align the light-diffusing section to be within axial confines of the inflatable balloon section.

37. A therapeutic system comprising the catheter system of claim 29 or the assembly of claim 33.

38. A method for PDT or VTP of a target site within a subject, comprising
systemically administering to the subject a photosensitizer drug;
inserting an assembly of claim 33 through a blood vessel and axially advancing and guiding it until the inflatable balloon section at the distal end portion of the balloon catheter is in the vicinity of the target site and axially displacing the optical fiber to position the light-diffusing section within said inflatable balloon section;
inducing a treatment stage comprising inflating the inflatable balloon section, passing a flushing solution through the flushing channel and irradiating light through the optical fiber at a wavelength and intensity suitable for activating the photosensitizer drug.

39. The method of claim 38, comprising
imaging the target site, and
positioning the light-diffusing section within said inflatable balloon section through proper alignment of the one or more fiber-associated imaging markers and the one or more balloon-associated imaging markers.

40. The method of claim 38, wherein the photosensitizer drug for use in the PDT procedure is a bacteriochlorophyll derivative having a major light absorption at about 750-756 nm and capable of generating oxygen radicals upon illumination, and
said photosensitizer drug is intravenously administered to the subject for a time period before inflation of the inflatable balloon section and the initiation of light irradiation, and wherein
the light source is a laser emitting light at a wavelength of about 750-756 nm with a power of up to about 2 Watts delivered to the fiber.

41. The method of claim 40, wherein said photosensitizer drug is Padeliporfin, i.e., WST11.

42. The method of claim 41, wherein the photosensitizer drug is administered about 5 to about 15 min. (typically about 10 min.) before light irradiation is initiated.

43. The method of claim 38, wherein
the target site is a tumor, and wherein
the blood vessel is a major vessel passing in the vicinity of the tumor.

44. The method of claim 43, wherein
the tumor is pancreatic cancer, and wherein
the blood vessel is the superior mesenteric artery (SMA), or superior mesenteric vein (SMV).

45. The method of claim 43, wherein the blood vessel is substantially tangential to the tumor.

46. The method of claim 38, comprising
two or more treatment stages, in which the inflatable balloon section is inflated and light irradiation is activated, separated by rest stages, in which the inflatable balloon section is deflated.

47. The method of claim 46, wherein
the treatment stages are for a period of about 2 to about 10 min., and wherein
the rest stages are for about 0.5 to about 2 min.

48. The method of claim 38, wherein the inflatable balloon section is inflated to substantially occlude blood flow through the blood vessel.
